(19) United States
(12) Patent Application Publication
(10) Pub. No.: US 2020/0147125 A1
(43) Pub. Date: May 14, 2020

(54) DETECTION OF HIGH RISK ARTERIAL THROMBOEMBOLIC DISEASES BY MARKERS OF COAGULATION AND HEMOSTATIC ACTIVATION

(71) Applicant: Fadi Basil NAHAB, Atlanta, GA (US)

(72) Inventor: Fadi Basil NAHAB, Atlanta, GA (US)

(21) Appl. No.: 16/627,865

(22) PCT Filed: Jul. 29, 2018
(86) PCT No.: PCT/US2018/044269

§ 371 Date: Dec. 31, 2019

Related U.S. Application Data
(60) Provisional application No. 62/538,995, filed on Jul. 31, 2017.

(51) Int. Cl. A61K 31/727; G01N 33/86; A61K 31/437; A61K 31/5377; A61K 31/4439; A61K 31/444; A61P 7/02

(52) U.S. Cl. CPC A61K 31/727; G01N 33/86; A61K 31/437; A61K 31/5377; G01N 2800/2871; A61K 31/444; A61P 7/02; G01N 2333/75; A61K 31/4439

(57) ABSTRACT

Disclosed herein is a method for identifying patients with cryptogenic stroke or embolic stroke of undetermined source (ESUS) who are at risk for having occult atrial fibrillation, underlying malignancy, and/or recurrent arterial thromboembolic events. Also disclosed is a method for determining who among these patients will benefit from anticoagulants. Also disclosed herein is a method to predict whether the anticoagulant is sufficiently effective to reduce the activated coagulation system in a patient. Also disclosed wherein is a method for identifying patients with cardiovascular and cerebrovascular arterial diseases who have occult atrial fibrillation, underlying but unrecognized malignancy, and/or are at high risk of recurrent arterial thromboembolic events.

### CROSS-REFERENCE TO RELATED APPLICATIONS

This application claims benefit of U.S. Provisional Application No. 62/538,995, filed Jul. 31, 2017, which is hereby incorporated herein by reference in its entirety.

### BACKGROUND

Around 15 million people worldwide suffer stroke each year, according to the World Health Organization. More than 30% of stroke victims die every year, while just over 30% suffer from permanent disability. This makes strokes the second most common cause of death and a major cause of disability. Strokes can affect people of all age groups, although it is most common in people aged over 65 years. WHO statistics show that for each decade of life after the age of 55, the rate of stroke is found to double for both males and females.

Approximately 800,000 strokes occur annually in the United States of which 88% are of ischemic origin. Previous studies have shown that ⅓ of ischemic strokes have no identified source of clot and are termed cryptogenic strokes or embolic strokes of undetermined source (ESUS). It is necessary to establish the cause of stroke to determine functional prognosis, decrease the risk of future strokes, and select appropriate preventative care.

### SUMMARY

Disclosed herein is a method for identifying patients with cryptogenic stroke or embolic stroke of undetermined source (ESUS) who are at risk for having occult atrial fibrillation, underlying malignancy, and/or recurrent arterial thromboembolic events. Also disclosed is a method for determining who among these patients will benefit from anticoagulants, such as apixaban, rivaroxaban, dabigatran, edoxaban, enoxaparin, warfarin, fondaparinux and heparin. Also disclosed herein is a method to predict whether the anticoagulant is sufficiently effective to reduce the activated coagulation system in a patient.

Also disclosed herein is a method for identifying patients with cardiovascular and cerebrovascular arterial diseases who have occult atrial fibrillation, underlying but unrecognized malignancy, and/or are at high risk of recurrent arterial thromboembolic events.

An assay for detecting coagulation activation, specifically for thromboembolic arterial diseases including cardiovascular and cerebrovascular arterial diseases such as ischemic stroke or transient ischemic attack, is disclosed that employs assaying D-dimers, fibrin monomer, thrombin-antithrombin complexes, and prothrombin fragment 1.2 (referred to as Markers of Coagulation and Hemostatic Activation (MOCHA) Panel). The disclosed methods involve comparing the level of the each test and the combination of four tests together with reference values. As disclosed herein, when intravascular thrombosis is suspected in the presence or absence of overt clinical symptoms, elevation in D-dimer, fibrin monomer, prothrombin fragment 1.2, and/or thrombin-antithrombin complex may be the only indicator of intravascular clot formation. Marked elevation in 2 or more of these markers should lead to further investigation for underlying high risk states for recurrent arterial thromboembolic disease including occult atrial fibrillation, malignancy and/or recurrent stroke.

In some embodiments, the disclosed MOCHA profile can be used in patients with arterial cardiovascular and cerebrovascular disease. In some embodiments, the disclosed MOCHA profile can be used in patients with cryptogenic stroke/embolic stroke of undetermined source (ESUS), an abnormal MOCHA profile identifies patients who are at increased risk of subsequent diagnosis of atrial fibrillation, malignancy, venous thromboembolism, or recurrent stroke. In some embodiments, the disclosed MOCHA profile can be used in patients with cryptogenic stroke/ESUS, patients with an abnormal MOCHA profile of ≥2 markers who are placed on anticoagulation have a significantly reduced risk of recurrent stroke compared to patients on antiplatelet therapy. In some embodiments, the disclosed MOCHA profile can be used in patients with congestive heart failure, an abnormal MOCHA profile identifying patients who may benefit from anticoagulation. In some embodiments, an abnormal MOCHA profile identifies patients who have a cardioembolic cause of TIA versus a TIA mimic (e.g. seizure). In some embodiments, a normal MOCHA profile identifies ischemic stroke patients who do not have an embolic mechanism of their stroke including radiation-induced vasculopathy, lacunar infarcts, large artery atherosclerotic occlusion, symptomatic intracranial atherosclerotic stenosis, arterial dissection, vasospasm. In some embodiments, abnormal MOCHA profile identifies patients who have an ischemic stroke with secondary hemorrhagic transformation vs those with primary intracerebral hemorrhage. In some embodiments, patients with abnormal MOCHA profile who are started on therapeutic levels of anticoagulation have normalization of their MOCHA levels. MOCHA profile abnormalities have been seen in patients with CML, prostate cancer, lymphoma, breast cancer, colon cancer, lung cancer, polycythemia vera, antiphospholipid antibody syndrome. In some embodiments, the disclosed MOCHA profile can identify a higher risk group of patients with atrial fibrillation despite risk stratification suggesting a low risk of stroke (e.g. low CHADSVASC scores) who may benefit from anticoagulation as opposed to antiplatelet therapy. In some embodiments, the disclosed MOCHA profile can be used to identify pregnant women who are at higher risk of thrombotic events. In some embodiments, the disclosed MOCHA profile can be used in patients with heart mechanical valves and/or left ventricular assist devices, an abnormal MOCHA profile despite routine anticoagulation can identify patients who are at higher risk for cardiovascular events and stroke. When the appropriate anticoagulation therapy range with warfarin and antiplatelet therapy is used, the MOCHA profile normalizes.

The details of one or more embodiments of the invention are set forth in the accompanying drawings and the description below. Other features, objects, and advantages of the invention will be apparent from the description and drawings, and from the claims.

### DETAILED DESCRIPTION

Disclosed herein is a method for treating a patient with cryptogenic stroke or embolic stroke of undetermined source (ESUS), comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject with a therapeutically effective amount of an anticoagulant. Non-limiting examples of anticoagulants include apixaban, rivaroxaban, dabigatran, edoxaban, enoxaparin, warfarin, fondaparinux and heparin.

Disclosed herein is a method for treating a patient with cryptogenic stroke or embolic stroke of undetermined source (ESUS), comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject with a therapeutically effective amount of an anticoagulant. Non-limiting examples of anticoagulants include apixaban, rivaroxaban, dabigatran, edoxaban, enoxaparin, warfarin, fondaparinux and heparin.

Also disclosed herein is a method for identifying patients with cardiovascular and cerebrovascular arterial diseases who have occult atrial fibrillation, underlying but unrecognized malignancy, and/or are at high risk of recurrent arterial thromboembolic events. As disclosed herein, elevated levels of one or more of d-dimer, F1.2, TAT complex, and fibrin monomer, is an indication that the patient is at high risk for having occult atrial fibrillation, an unrecognized malignancy and/or is at high risk of recurrent arterial thromboembolic events.

Therefore, also disclosed herein is a method for treating a patient with cardiovascular and cerebrovascular arterial diseases, comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject with a therapeutically effective amount of a chemotherapy, immunotherapy, radiotherapy, anticoagulation including apixaban, rivaroxaban, dabigatran, edoxaban, enoxaparin, warfarin, fondaparinux, heparin or any combination thereof. Normalization of d-dimer, prothrombin fragment 1.2 (F1.2), thrombin-antithrombin (TAT) complex, and fibrin monomer levels would indicate a therapeutically effective amount of a chemotherapy, immunotherapy, radiotherapy, or anticoagulant.

Atrial fibrillation (AF) is a very common arrhythmia and significantly increases stroke risk. This risk can be mitigated with oral anticoagulation, but AF is often asymptomatic, or occult, preventing timely detection and treatment. Accordingly, occult AF may cause stroke before it is clinically diagnosed. Also disclosed herein is a method for treating a patient with cardiovascular and cerebrovascular arterial diseases, comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject for occult atrial fibrillation, e.g. with anticoagulation therapy including apixaban, rivaroxaban, dabigatran, edoxaban, enoxaparin, warfarin, fondaparinux or heparin.

As disclosed herein, when intravascular thrombosis is suspected in the absence of overt clinical symptoms, elevation in D-dimer, fibrin monomer, prothrombin fragment 1.2, and/or thrombin-antithrombin complex may be the only indicator of intravascular clot formation. Therefore, also disclosed herein is a method for treating a patient with cardiovascular and cerebrovascular arterial diseases, comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and further investigating the subject for the site of the thrombosis. The method can further involve treating the subject for thrombosis, e.g. with thrombectomy, vasodilatory therapy, thrombolytic therapy, or anticoagulation therapy.

Measure of Coagulation and Hemostatic Activation (MOCHA) Panel

The MOCHA panel is a group of 4 blood tests that include: a d-dimer, prothrombin fragment 1.2, thrombin-antithrombin complex, and fibrin monomer. These tests measure elements of the blood coagulation cascade and when combined provide advantages over the individual blood test results.

D-dimer is a fibrin degradation product (or FDP), a small protein fragment present in the blood after a blood clot is degraded by fibrinolysis. It is so named because it contains two D fragments of the fibrin protein joined by a cross-link. D-dimer concentration may be determined by a blood test to help diagnose thrombosis. Reference ranges for D-dimer in non-pregnant adults is less than or equal to 287 ng/mL.

Prothrombin fragment 1.2 (F1.2) is a peptide released when prothrombin is cleaved by factor Xa. This fragment binds to phospholipid through calcium and interacts with factor Va. Elevated plasma levels of F1.2 have been described in patients with thrombosis or in prethrombotic states. F1.2 levels are useful for diagnosing hypercoagulable state and as a marker for disseminated intravascular coagulation (DIC). The reference range for F1.2 is 65-288 pmol/L.

Thrombin-antithrombin (TAT) complex is a parameter of coagulation and fibrinolysis. Elevated concentrations have been associated with vascular complications associated with diabetes. The reference range for TAT is 1.0-5.5 mcg/L.

The Fibrin Monomer test is typically used in diagnosing DIC or in differentiating DIC from primary fibrinolysis. Fibrin Monomer may be useful in monitoring response to treatment of DIC. The reference range for fibrin monomer is less than 7 mcg/mL.

The d-dimer, F1.2, TAT complex, and fibrin monomer proteins can be detected in samples from subjects using, for example, an immunoassay. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Methods

ESUS patients were identified who underwent prolonged cardiac monitoring with mobile cardiac outpatient telemetry (MCOT) and/or implantable loop recorder (ILR). In a subset of consecutive ESUS patients, 4 measures of coagulation and hemostatic activation (MOCHA) including d-dimer, prothrombin fragment 1.2, thrombin-antithrombin complex and fibrin monomer were obtained ≥2 weeks after the index stroke and repeated if abnormal. The ability of of abnormal MOCHA levels to identify patients who had the composite outcome of newly diagnosed AF, malignancy, or recurrent stroke were evaluated.

Results

During the study period, 92 ESUS patients (mean age 64 +/−15 years, 54% female, 62% non-white, mean follow-up 1.4 +/−0.8 years) underwent prolonged cardiac monitoring (65% MCOT, 62% ILR, 38% MCOT+ILR); 16 (17%) were found to have AF during follow-up. Baseline characteristics of ESUS patients who underwent MOCHA testing (n=44) were similar to patients who did not except that those tested were younger (60 vs 67 years, p=0.04); over a mean follow-up of 1.2 +/−0.8 years, 18 (41%) patients were newly diagnosed with AF, malignancy or recurrent stroke. ESUS patients with persistently abnormal (vs normal) MOCHA levels were significantly more likely to have newly diagnosed AF, malignancy, or recurrent stroke (OR 11.3, 95% CI 2.5-50.1, p=0.001). Elevated levels of ≥3 MOCHA markers had a 67% sensitivity and 81% specificity for identifying patients subsequently diagnosed with AF, malignancy, or recurrent stroke.

Conclusion

Abnormal MOCHA levels identified ESUS patients who were more likely to have newly diagnosed AF, malignancy or recurrent stroke over follow-up and are effective in identifying patients who could benefit from early anticoagulation.

### Example 2

Background

Current medical management of cryptogenic stroke/ESUS recommends antiplatelet monotherapy though some causes of cryptogenic stroke may benefit from anticoagulation (e.g. occult atrial fibrillation or occult malignancy). Serum markers of coagulation and hemostatic activation (MOCHA) have previously been associated with venous thrombosis however there is limited data in patients with arterial disease. The MOCHA profile consists of 4 markers:

D-dimer (reference range <500 ng/mL)

Fibrin monomer (reference range <7 mcg/mL)

Prothrombin fragment 1.2 (reference range 65-288 pmol/L)

Thrombin-Antithrombin complex (reference range 1.0-5.5 mcg/L)

D-dimer represents a marker of fibrinolysis and a byproduct of fibrin degradation. Fibrin monomer (also known as soluble fibrin) is a marker of coagulation activation and a byproduct of fibrinogen conversion to fibrin. Prothrombin fragment 1.2 (F 1+2) is a marker of coagulation activation and a peptide released during conversion of prothrombin to thrombin. Thrombin-antithrombin complex (TAT) is a marker of coagulation activation and is a complex formed during thrombin formation.

The MOCHA profile was incorporated into the disclosed hypercoagulable testing of patients. 42 consecutive patients were evaluated in a pilot study evaluating the utility of the MOCHA profile in patients with cryptogenic stroke/ESUS. Baseline characteristics are provided in Table 1.

TABLE 1

| | MOCHA tested | |
|---|---|---|
| Characteristics | N = 42 | |
| Demographics | | |
| Age, mean (SD) | 60 | (17) |
| Female, n (%) | 26 | (62%) |
| Race, n (%) | 24 | (56%) |
| Non-white | | |
| BMI, mean (SD) | 28.8 | (7.6) |
| Comorbidities, n (%) | | |
| Hypertension | 30 | (71%) |
| Diabetes Mellitus | 18 | (43%) |
| Hyperlipidemia | 25 | (60%) |
| Coronary Artery Disease | 3 | (7.1%) |
| Previous ischemic stroke | 4 | (9.5%) |
| Tobacco (former or active) | 11 | (26%) |
| ILR | 28 | (67%) |
| Follow-up duration, mean years (SD) | 1.1 | (0.6) |

Results showed that an abnormal MOCHA profile as defined as ≥2 abnormal markers was associated with an increased risk of subsequent new diagnosis of atrial fibrillation, malignancy or recurrent stroke compared to patients with a normal MOCHA profile.

The data showed that a normal MOCHA profile had a 100% sensitivity for identifying patients who were not subsequently diagnosed with atrial fibrillation, malignancy or recurrent stroke. In patients with ≥2 abnormal markers in their MOCHA profile, there was a positive predictive value of 0.50 for detecting subsequent atrial fibrillation, malignancy or recurrent stroke.

A validation study was performed to evaluate the predictive ability of the MOCHA profile to identify patients with subsequent diagnosis of atrial fibrillation, malignancy, stroke or the composite outcome. Results are shown in Table 2. Additionally, patients with an abnormal MOCHA profile (≥2 markers) were offered the option of anticoagulation therapy instead of antiplatelet therapy. In the validation study, an abnormal MOCHA again effectively predicted the subsequent diagnosis of atrial fibrillation or malignancy in patients. Most importantly, anticoagulation of patients with an abnormal MOCHA profile significantly reduced the risk of recurrent stroke from 14% in the pilot study down to 0.9% (p=0.0004). In both the pilot and validation studies, no patients with a normal MOCHA placed on antiplatelet therapy had a recurrent stroke.

TABLE 2

| | Initial | | Validation | | |
|---|---|---|---|---|---|
| | cohort | | cohort | | P- |
| Characteristics | N = 42 | | N = 113 | | value |
| Demographics | | | | | |
| Age, mean (SD) | 60 | (17) | 64 | (15) | 0.099 |
| Female, n (%) | 26 | (62%) | 71 | (63%) | 0.92 |
| Race, n (%) | 24 | (56%) | 61 | (54%) | 0.777 |
| Non-white | | | | | |
| Comorbidities, n (%) | | | | | |
| Hypertension | 30 | (71%) | 89 | (79%) | 0.517 |
| Diabetes | 18 | (43%) | 26 | (23%) | 0.004 |
| Previous ischemic stroke | 4 | (10%) | 11 | (10%) | 1 |
| Tobacco (former or active) | 11 | (26%) | 45 | (40%) | 0.101 |
| ILR | 28 | (67%) | 47 | (42%) | 0.0003 |
| Follow-up duration, mean years | 1.1 | (0.6) | 7 | (4) | 0.0001 |
| (SD) | | | | | |
| MOCHA (≥2 abnormal) | 23 | (55%) | 41 | (36%) | 0.008 |
| AFib | 6 | (14%) | 6 | (5.3%) | 0.037 |
| Malignancy | 4 | (9.5%) | 9 | (8%) | 0.708 |
| VTE | 0 | (0%) | 8 | (7.1%) | 0.007 |
| AF, Maligancy, VTE, other | 14 | (33%) | 30 | (26.5%) | 0.427 |
| hypercoagulable state | | | | | |
| Recurrent Stroke | 6 | (14%) | 1 | (0.9%) | 0.0004 |
| Major Hemorrhage | 0 | (0%) | 0 | (0%) | 1 |

All patients in the validation study placed on anticoagulation had normalization of their abnormal MOCHA levels after treatment. Additionally, all malignancies detected in the validation cohort were associated with abnormal MOCHA levels including prostate cancer (n=2), breast cancer (n=1), colon cancer (n=1), bladder cancer (n=1), renal cancer (n=1) and polycythemia vera (n=1).

Summary

The MOCHA profile when abnormal in ≥2 markers identify patients who are at increased risk of subsequent diagnosis of atrial fibrillation, malignancy or recurrent stroke. The MOCHA profile is effective as a guide to determine which patients benefit from anticoagulation therapy, resulting in a reduced risk of recurrent stroke. The normalization of the MOCHA profile after patients are placed on anticoagulation therapy provides additional support for treatment of the hypercoagulable state and can be used in other arterial cardiovascular indications including patients with mechanical heart valves or left ventricular assist devices. Results show that a normal MOCHA profile is associated with low risk of future stroke in patients with cryptogenic stroke/ESUS, congestive heart failure, atrial fibrillation and pregnancy.

Additionally, the MOCHA profile distinguishes cardiac causes of arterial thrombosis including atrial fibrillation and congestive heart failure among other causes. This can be useful to differentiate cardioembolic causes of TIA from TIA mimics, distinguishes causes of cardioembolic versus non-cardioembolic causes of ischemic and hemorrhagic strokes. Results also show that an abnormal MOCHA profile can be seen in patients with a variety of malignancies and hypercoagulable states including chronic myelocytic leukemia, prostate cancer, lymphoma, breast cancer, colon cancer, lung cancer, polycythemia vera and antiphospholipid antibody syndrome.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a patient with cryptogenic stroke or embolic stroke of undetermined source (ESUS), comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject with a therapeutically effective amount of an anticoagulant.

2. The method of claim 1, wherein the anticoagulant comprises apixaban, rivaroxaban, dabigatran, edoxaban, enoxaparin, warfarin, fondaparinux, heparin, or any combination thereof.

3. A method for treating a patient with cardiovascular and cerebrovascular arterial diseases due to an occult malignancy, comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject with anti-cancer therapy.

4. The method of claim 3, wherein the anti-cancer therapy is selected from the group consisting of chemotherapy, immunotherapy, and radiotherapy.

5. A method for treating a patient with cardiovascular and cerebrovascular arterial diseases, comprising assaying a sample from the subject for levels of a d-dimer, prothrombin fragment 1.2 (F1.2), thrombin-antithrombin (TAT) complex, and fibrin monomer, detecting elevated levels of one or more of d-dimer, F1.2, TAT complex, and fibrin monomer, and treating the subject with oral anticoagulation therapy, investigating the subject for the site of a thrombosis, or a combination thereof.

6. (canceled)

7. The method of claim 5, further comprising treating the subject for thrombosis.

8. The method of claim 7, wherein the subject is treated with thrombectomy, vasodilatory therapy, thrombolytic therapy, anticoagulation therapy, or any combination thereof.
